(19) United States
(12) Patent Application Publication
(10) Pub. No.: US 2024/0041411 A1
(43) Pub. Date: Feb. 8, 2024

(54) BIO-SIGNAL MEASURING DEVICE AND METHOD OF USING THE SAME

(71) Applicant: ATSENS CO., LTD., Gyeonggi-do (KR)

(72) Inventors: Seung Bum CHO, Gyeonggi-do (KR); Jong Ook JEONG, Gyeonggi-do (KR)

(73) Assignee: ATSENS CO., LTD., Gyeonggi-do (KR)

(21) Appl. No.: 18/357,321

(22) Filed: Jul. 24, 2023

(30) Foreign Application Priority Data
Aug. 2, 2022 (KR) 10-2022-0096108

(51) Int. Cl. A61B 5/00; A61B 5/0205

(52) U.S. Cl. CPC A61B 5/7285; A61B 5/6802; A61B 5/0205

(57) ABSTRACT

Provide are a bio-signal measuring device capable of maintaining a contact force when attaching sensors for measuring bio-signals to the skin and a method of using the same. The bio-signal measuring device includes a housing accommodating a battery, and a flexible substrate extending in a band shape, disposed on one side of the housing and electrically connected thereto, and including a plurality of sensor electrodes. The housing and the plurality of sensors are disposed along a longitudinal direction of the substrate. The housing is disposed between the plurality of sensors at a position spaced apart from a center of the substrate.

### CROSS-REFERENCE TO RELATED APPLICATION

This application is based on and claims priority under 35 U.S.C. § 119 to Korean Patent Application No. 10-2022-0096108, filed on Aug. 2, 2022, in the Korean Intellectual Property Office, the disclosure of which is incorporated by reference herein in its entirety.

### BACKGROUND

The present disclosure relates to a bio-signal measuring device for measuring bio-signals such as an electrocardiogram signal and using the same for health management and a method of using the bio-signal measuring device.

As the general public's interest in health management has increased due to improvement of the economic level, aging of the population, increasing interest in health management for various health conditions through physical activities and regular sports activities has increased. To carry out health management, it is necessary to develop a bio-signal measuring device equipped with a sensor for measuring bio-signals, such as electrocardiogram (ECG), respiration, heart pulse wave, and electromyogram (EMG) related to the movement of muscles in the body, at all times.

For example, the ECG, which may be used as representative biometric information, records the action electric potential generated as cardiac muscle contracts and expands due to heartbeat of a user. A method of measuring the ECG includes attaching electrodes to the skin of the user to measure active voltages according to the contraction of the cardiac muscle, and then depicting the measured data as a waveform.

In particular, the action potential generated when the cardiac muscle contracts and relaxes due to the heartbeat is transmitted from the heart to the whole body and generates a potential difference depending on the position of the body. Then, this potential difference may be detected and recorded through surface electrodes attached to the skin of the user.

The ECG is used to check for abnormalities in the heart, and is used as a basic measurement method for diagnosing cardiac diseases such as angina pectoris, myocardial infarction, and arrhythmia.

As described above, to measure the ECG, an ECG sensor electrode must maintain close contact with the skin, and this also applies to the EMG sensor electrode for measuring the EMG and a sensor for measuring a respiration signal. However, there are problems in that the quality of the measured signal is low due to a decrease of the adhesive strength over time, the curved shape of the human body, and the limitations of adhesives applicable to the human body. Therefore, there is a need for a bio-signal measuring device capable of maintaining a contact force when attaching the sensors to the skin.

The above-mentioned background art is technical information that the inventor has to derive the present disclosure or has been acquired in the process of deriving the present disclosure, and cannot necessarily be considered as a known technique disclosed to the general public prior to the filing of the present application.

### SUMMARY

The present disclosure provides a bio-signal measuring device capable of maintaining a contact force when attaching sensors for measuring bio-signals to the skin and a method of using the same.

However, these problems are just examples, and the problems to be solved by the present disclosure are not limited thereto.

Embodiments of the present disclosure provide the following bio-signal measuring device and its use method to solve the above problems.

According to an aspect of the present disclosure, there is provided a bio-signal measuring device including a housing accommodating a battery, and a flexible substrate extending in a band shape, disposed on one side of the housing and electrically connected thereto, and including a plurality of sensor electrodes, wherein the housing and the plurality of sensors are disposed along a longitudinal direction of the substrate, wherein the housing is disposed at any position of the substrate but is usually disposed at a position spaced apart from a center of the substrate.

The bio-signal measuring device may further include at least one adapter formed to correspond to the housing and the substrate and disposed to press the substrate.

The adapter may include a first pressing part disposed on one side of the substrate, a second pressing part disposed on another side of the substrate, and a connection part connecting the first pressing part to the second pressing part, wherein a housing groove into which the housing is inserted may be formed between the first pressing part and the second pressing part.

A thickness of the housing groove side end of the first pressing part and the second pressing part may correspond to a housing thickness.

A thickness of outer ends of the first pressing part and the second pressing part may gradually decrease.

A shape of the upper end of the first pressing part and the second pressing part may be in a straight line.

The connection part may be formed at a lower part of the housing groove.

The bio-signal measuring device may further include an elastic band including an elastic material to press the substrate or the adapter.

The elastic band may include a buckle device or Velcro fabric at both ends, so that both ends of the elastic band are connected and detachable.

The sensor may include a capacitance sensor installed in the housing and detecting a respiration signal by sensing respiration and an ECG sensor installed in the substrate and generating an ECG..

The bio-signal measuring device may further include an alarm unit that issues an alarm through a lamp or speaker when a magnitude of the respiration signal or ECG signal decreases.

The bio-signal measuring device may further include at least one of an acoustic sensor for detecting snoring or respiration during sleep and an acceleration sensor for detecting movement.

According to another aspect of the present disclosure, there is provided a bio-signal measuring device including a housing accommodating a battery, a flexible substrate extending in a band shape, disposed on one side of the housing and electrically connected thereto, and including a plurality of electrodes, and an adapter formed to correspond to the housing and the substrate and disposed to press the substrate.

According to another aspect of the present disclosure, there is provided a method of using a bio-signal measuring device, the method including attaching a substrate including respiration and ECG measurement sensors to a level near a user's lung, and placing a band including an elastic material to press an outer surface of the substrate toward the user's lung. The method may further include disposing an adapter formed in a shape corresponding to the substrate on an outer surface of the substrate before the placing the band.

Other aspects, features, and advantages other than those described above will become clear from the detailed description, claims, and drawings for carrying out the invention below.

### BRIEF DESCRIPTION OF THE DRAWINGS

Embodiments will be more clearly understood from the following detailed description taken in conjunction with the accompanying drawings in which:

FIG. 1 is a perspective view of an embodiment of a bio-signal measuring device according to the present disclosure;

FIG. 2 is a plan view of FIG. 1;

FIG. 3 is a longitudinal cross-sectional view taken along line III-III′ in FIG. 2;

FIG. 4 is an exploded perspective view of FIG. 1;

FIG. 5 is an exploded perspective view of the measuring device in FIG. 4;

FIG. 6 is a diagram showing a method of using the bio-signal measuring device of the present disclosure; and

FIG. 7 is a flowchart of an embodiment of a method of using the bio-signal measuring device of the present disclosure.

### DETAILED DESCRIPTION OF THE EMBODIMENTS

Since the present disclosure may apply various modifications and have various embodiments, certain embodiments are illustrated with reference to the drawings and will be described in detail. However, this is not intended to limit the present disclosure to certain embodiments, and should be understood to include all modifications, equivalents and substitutes included in the spirit and scope of the present disclosure. In the description of the present disclosure, even though illustrated in other embodiments, like reference numerals refer to like components.

Hereinafter, embodiments of the present disclosure will be described in detail with reference to the accompanying drawings, and when describing with reference to the drawings, the same or corresponding constituent elements are given the same reference numerals, and redundant descriptions thereof will be omitted.

In the following embodiments, terms such as first and second are not used in a limiting meaning, but for the purpose of distinguishing one component from another component.

In the following examples, the singular expression includes the plural expression unless the context clearly indicates otherwise.

In the following embodiments, terms such as include or have means that the features or elements described in the specification are present, and do not preclude the possibility of adding one or more other features or elements in advance.

In the drawings, components may be exaggerated or reduced in size for convenience of description. For example, the size and thickness of each component shown in the drawings are arbitrarily shown for convenience of description, and thus the present disclosure is not necessarily limited to what is shown.

In the following embodiments, the x-axis, the y-axis, and the z-axis are not limited to three axes on a Cartesian coordinate system, and may be interpreted in a broad sense including them. For example, the x-axis, y-axis, and z-axis may be orthogonal to each other, but may refer to different directions that are not orthogonal to each other.

When a certain embodiment may be implemented differently, a particular process order may be performed differently from the described order. For example, two processes described in succession may be performed substantially simultaneously, or may be performed in an order opposite to the described order.

The terms used in the present application are used only to describe certain embodiments, and are not intended to limit the present disclosure. In this specification, the term “include,” “comprise,” “including,” or “comprising,” specifies a property, a region, a fixed number, a step, a process, an element and/or a component but does not exclude other properties, regions, fixed numbers, steps, processes, elements and/or components.

Hereinafter, certain embodiments of the present disclosure will be described in detail with reference to the drawings.

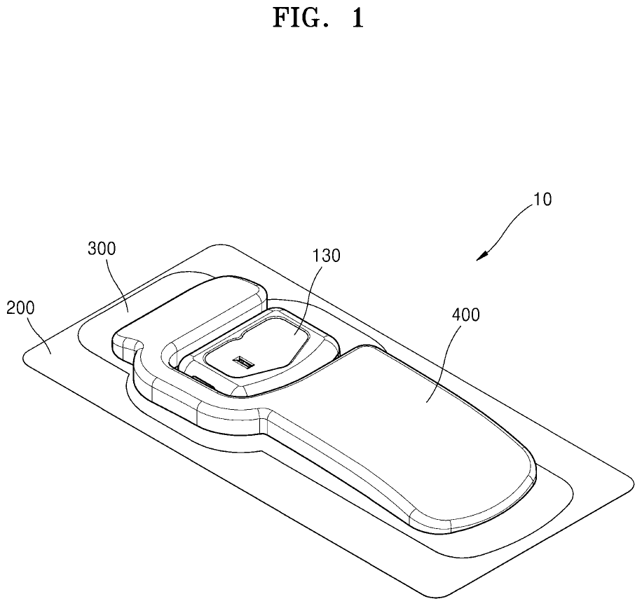

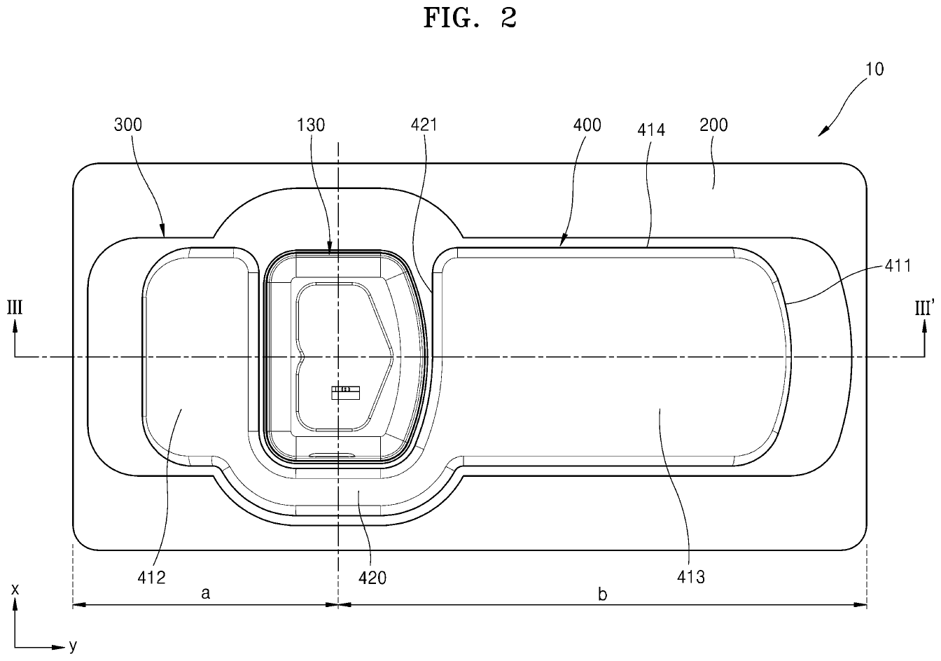

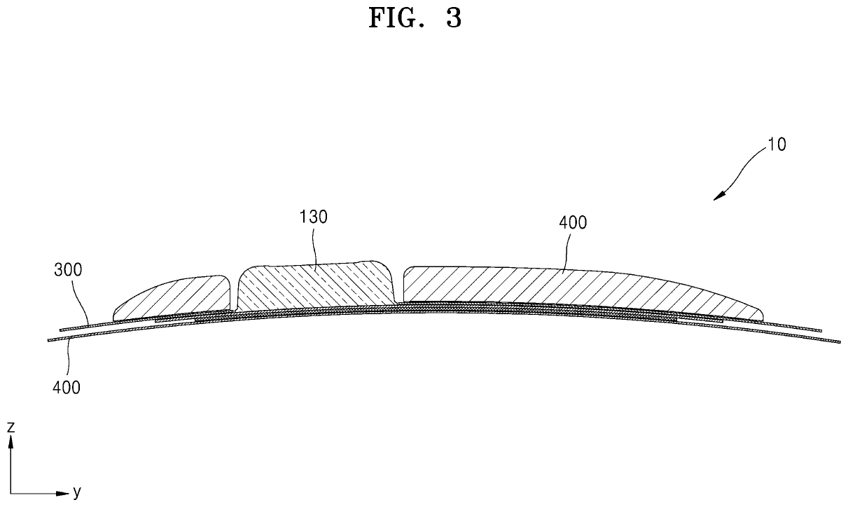

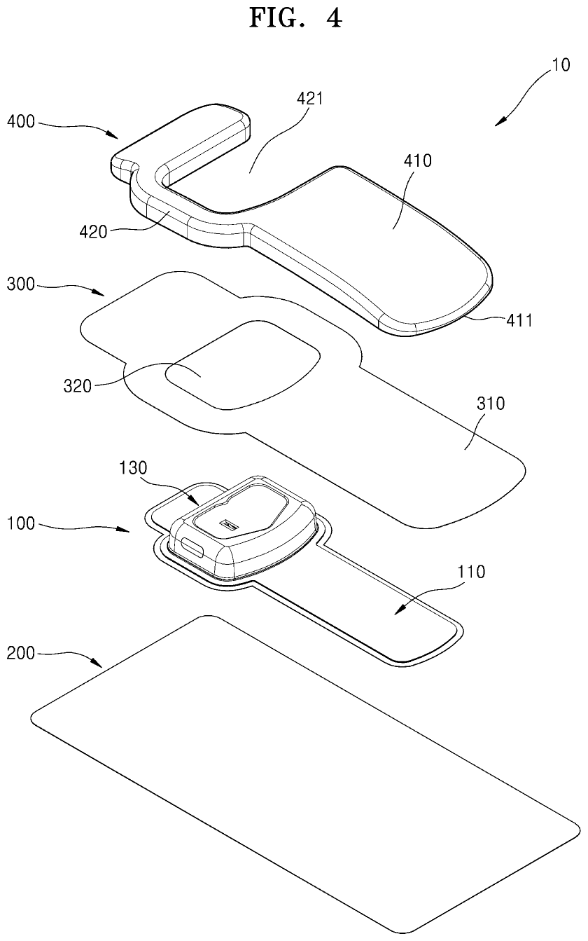

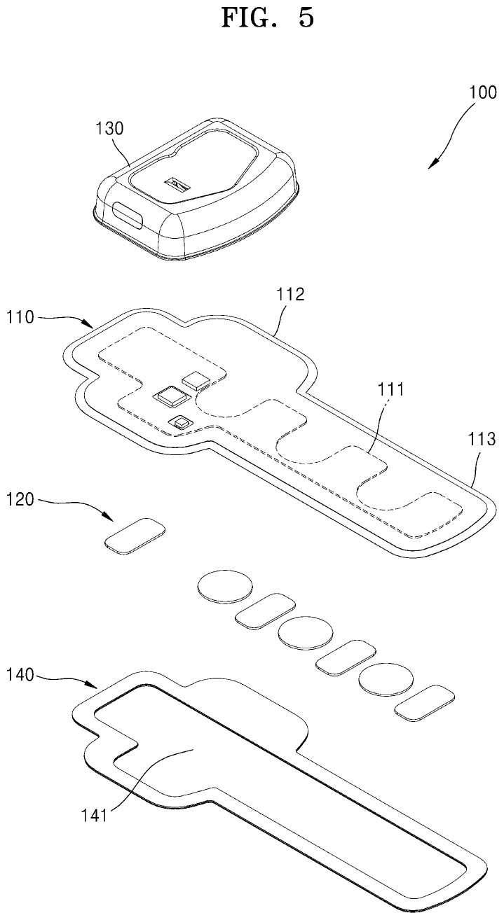

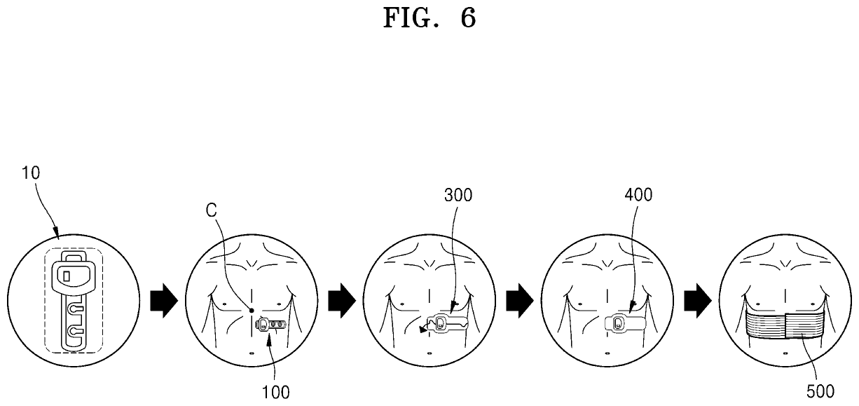

FIG. 1 is a perspective view of an embodiment of a bio-signal measuring device 10 of the present disclosure, FIG. 2 is a plan view of FIG. 1, FIG. 3 is a longitudinal cross-sectional view taken along line III-III′ in FIG. 2, FIG. 4 is an exploded perspective view of FIG. 1, FIG. 5 is an exploded perspective view of the measuring device 100 in FIG. 4, and FIG. 6 is a diagram illustrating a method of using the bio-signal measuring device 10 of the present disclosure.

As shown in these drawings, one embodiment of the bio-signal measuring device 10 of the present disclosure includes a measuring device 100 equipped with a sensor electrode 120 (see FIG. 5) and worn to closely adhere to the skin. Furthermore, the bio-signal measuring device 10 may further include a release film 200 attached to one side of the measuring device 100, a sub-patch 300 disposed on the other side of the measuring device 100, an adapter 400 disposed on the other side of the measuring device 100 to press the measuring device 100, and an elastic band 500 (shown in FIG. 5) for fixing the adapter 400 and strengthening the degree of adhesion of the measuring device 100.

The measuring device 100 supplies electricity to a plurality of semiconductors and receives signals from the sensor electrodes 120, and circuits are formed on the first substrate 110. In addition, the measuring device 100 may be formed of a flexible printed circuit board (FPCB) to correspond to a curved skin surface. Furthermore, the measuring device 100 is composed of a plurality FPCBs, and may be formed in a form in which a second substrate 111, which is a general printed circuit board (PCB), is coupled to a first substrate 110, which is an FPCB. As shown in FIG. 5, the second substrate 111 may be formed in a thin bridge shape where the sensor electrode 120 is not attached so that the second substrate 111 may be bent well.

A housing arrangement part 112 is formed on one side of the measuring device 100, and a substrate extension part 113 extends in a strip shape in the lateral direction of the housing arrangement part 112, so that the measuring device 100 may have a longitudinal shape as a whole. Furthermore, the substrate extension part 113 may be formed on both sides of the housing arrangement part 112. In this case, the housing arrangement part 112 may be formed at a position spaced apart from the center of the measuring device 100, not the center of the measuring device 100. That is, substrate extension parts 113 extending from both sides of the housing arrangement part 112 may have different lengths. As shown in FIG. 2, a distance ‘a’ from the housing to one side and a distance ‘b’ to the other side may be formed differently.

Wearing comfort of a wearer may be improved, when attaching the bio-signal measuring device 10 of the present disclosure to a position slightly spaced apart from the center of the human body to the left. First, when the bio-signal measuring device 10 of the present disclosure slightly moved from the center of the chest, it may be difficult to sense an electrocardiogram (ECG) signal due to a small potential difference. Therefore, it is useful to move the location of the bio-signal measuring device 10 of the present disclosure from the center to the outside more than the location of the heart. However, in this case, it is difficult to adhere closely due to the curved shape of the chest. Therefore, according to the bio-signal measuring device 10 of the present disclosure, the bulky housing 130 is eccentrically disposed to one side, and the narrow substrate extension part 113 is disposed immediately below the bust point.

A sensor electrode 120 as shown in FIG. 5 may be attached to one surface of the PCB. The sensor electrode 120 may be installed in the housing 130 as well. Additionally or alternatively, a plurality of sensor electrodes 120 may be disposed along the extension direction of the housing 130 and the measuring device 100. A housing arrangement part 112 may be formed between the plural sensor electrodes 120, as shown in FIG. 5.

The sensor electrode 120 may measure bio-signals, and may include, for example, a respiration sensor, an ECG sensor, and an electromyogram (EMG) sensor. In some embodiments, a respiration sensor is a sensor that detects capacitance change due to breathing activity. An ECG sensor is a sensor that senses a potential difference according to contraction and expansion of the heart using an ECG electrode. Heart rate, arrhythmia, and the like may be detected using the sensed potential difference. An EMG sensor measures a potential difference between EMG electrodes like an ECG electrode, and may detect an electrical signal required for muscle movement. In addition, a snoring sensor, a toss and turn sensor, a temperature sensor and an impedance sensor may be further included. The snoring sensor is an acoustic sensor and is a sensor that recognizes a snoring sound and an apnea state. The toss and turn sensor may include an acceleration sensor or a gyro sensor. The acceleration sensor senses a change in momentum of an object and can detect a change in momentum of the measurement device 100. The toss and turn sensor may detect a change in the posture of the user. The toss and turn sensor may measure the roll-over degree according to the degree of posture change.

Meanwhile, the respiration sensor among the plurality of sensor electrodes 120 needs to be in close contact with the human body of a wearer. Therefore, the respiration sensor may be installed in the housing 130, which is easier to adhere to the skin of the wearer than the measuring device 100 when pressed. The respiration sensor is a capacitance sensor, which measures the change in capacitance as the volume of the chest expands and contracts during breathing activity. The number of respirations per time may be calculated using the capacitance change period. The respiration sensor electrode has an insulator between the electrode and the skin to measure the capacitance changed by respiration.

The bio-signal measuring device 10 of the present disclosure may include an alarm unit in the housing 130 or the measuring device 100. The alarm unit may be controlled to generate a visual or audible alarm through a lamp or a speaker when the level or frequency of the respiration signal or the ECG signal decreases.

In addition, a main patch 140 may be attached to one surface of the measuring device 100. The main patch 140 is a configuration for attaching the measuring device 100 to the human body, and may be formed in a shape corresponding to the measuring device 100. However, a hole 141 may be formed through a portion where the sensor electrode 120 is disposed so that the sensor electrode 120 may conduct electricity with the skin.

In addition, the housing 130 may be disposed and fixed to the other surface of the measuring device 100. The housing 130 may be installed on the housing arrangement part 112 on the measuring device 100. The housing 130 may include a communication unit that collects information measured from the battery and the sensor electrode 120 and transmits the collected information to the monitoring device. The communication unit may include an identification (ID) for Bluetooth connection. Furthermore, the present disclosure may further include an alarm unit and a control unit for controlling all of the electrical components described above.

The housing 130 may be formed of a plastic injection molding material, and has a preset thickness due to a battery or the like being installed therein. Therefore, since the housing 130 protrudes more than other parts of the measuring device 100, the housing 130 has a structure that is more easily adhered to the human body when pressed toward the human body. Therefore, the respiration sensor electrode 120 may be installed in the housing 130.

A release film 200 may be disposed on one surface of the measuring device 100 to protect the adhesion of the main patch 140. Therefore, the bio-signal measuring device 10 of the present disclosure may be supplied with the release film 200 attached to the main patch 140, and it is useful to remove the release film 200 immediately before use.

As described above, the plurality of sensor electrodes 120 and the housing 130 including a battery are disposed in the measuring device 100 formed of one or more, and a main patch 140 is attached to the lower surface of the measuring device 100 so that the measuring device 100 may be attached to the human body.

A sub-patch 300 may be further provided on the upper surface of the measuring device 100 to further provide the human body adhesion, as shown in FIG. 4. The sub-patch 300 may be formed of a film having adhesive strength. The patch body may be formed to correspond to the shape of the first substrate 110. In particular, a patch body 310 corresponding to the shape of the measuring device 100 and the housing 130 may be formed, and a patch hole 320 may be formed through a point where the housing 130 is disposed. The housing 130 may pass through the patch hole 320. The patch body 310 may be formed larger than the width and thickness of the measuring device 100 so as to be attached to the human body while covering the upper surface of the measuring device 100.

Due to the above configuration, the measuring device 100 and the sub-patch 300 may be formed in a flexible material and a flexible shape as a whole.

Therefore, if the upper part of the measuring device 100 or the sub-patch 300 is provided using the main patch 140 and the sub-patch 300, an adapter 400 may be disposed above the sub-patch 300. The adapter 400 is formed to correspond to the housing 130 and the measuring device 100, and is formed and arranged to press the measuring device 100 toward the human body. The adapter body 410 may be formed of a hard material such as a plastic injection molding.

As shown in FIG. 2, the adapter body 410 includes a first pressing part 412 disposed on one side of the housing 130 and a second pressing part 413 disposed on the other side of the housing 130, and the first pressing part 412 and the second pressing part 413 may be formed in a shape connected through the connection part 420. The connection part 420 may be formed to protrude outward to avoid interference with the housing 130. In particular, the connection part 420 may be formed below the housing groove 421. That is, when the adapter 400 is worn, the connection part 420 may be formed to be located on the lower part of the navel side, not the chest side. This is to avoid interference with the chest.

Therefore, a space defined by the housing groove 421 is formed between the first pressing part 412 and the second pressing part 413, and the housing 130 may be positioned in the housing groove 421 when worn by a wearer. The thickness of the ends of the first pressing part 412 and the second pressing part 413 on the side of the housing groove 421 may correspond to the thickness of the housing 130. Accordingly, since the protruding heights of the adapter 400 and the housing 130 are the same or similar, pressure may be uniformly applied when force is applied toward the human body. An adhesive or an adhesive film is attached to the concave surface of the adapter 400 to be attached to the measuring device 100 or the sub-patch 300, and a tape may be attached to protect the adhesive or adhesive film. It is useful to remove the tape immediately before application.

The adapter body 410 may have a concave shape on a surface facing the measuring device 100 so as to uniformly pressurize a curved human body. As shown in the drawing, it is also possible that the entire adapter body 410 is formed in a curved shape.

In addition, the adapter body 410 may be formed such that the thicknesses of the side ends 411 of the first pressing part 412 and the second pressing part 413 gradually decrease. Therefore, the central side where the housing groove 421 is formed may be thick and become thinner towards both ends. This is to reduce discomfort caused by pressure on the edges of both ends when the adapter 400 is worn. Also, the upper end 414 of the adapter body 410 may be formed in a straight line. This is a shape to reduce interference with the chest when worn.

In addition, an elastic band 500 may be included as an embodiment. The elastic band 500 is for pressing the measuring device 100 or the adapter 400 toward the human body, and may be formed of an elastic material to increase the pressing force. The elastic band 500 is for pressing the measuring device 100 or the adapter 400 toward the human body, and may be formed of an elastic material to increase the pressing force. Furthermore, the adapter 400 may be embedded into the elastic band 500 or integrated into the elastic band 500.

FIG. 6 shows one embodiment using the bio-signal measuring device 10 according to the present disclosure described above. Referring to FIG. 6, the patch attachment area is first checked before wearing the bio-signal measuring device 10 of the present disclosure. The patch attachment area is a part of the lung C, and more particularly, the measuring device 100 is positioned on the rib below the left breast at a height similar to the lung C. Afterwards, if there is a lot of hair in the patch attachment area, remove the hair with a razor, wash the patch attachment area with an alcohol swab, and dry the patch attachment area sufficiently for at least 1 minute.

Then, as shown in FIGS. 4-5, the release film 200 attached to the main patch 140 is removed, and the measuring device 100 is attached to the attachment area. In this case, it is necessary to press the entire area evenly for at least 1 minute so that the patch adheres well. Then, the battery is energized and connected to a device capable of monitoring and control such as a smartphone app to check the ECG signal and respiration signal. Connection with a smartphone or the like may use a Bluetooth method. Thereafter, the sub-patch 300 is attached on the measuring device 100, the protective film on the sub-patch 300 is removed, the tape of the adapter 400 is removed, and the guide pad is attached on the sub-patch 300. Then, an elastic band 500 is worn at a position covering the measuring device 100, as shown in FIG. 6. At this time, the elastic band 500 should be worn in an extended state so that the measuring device 100 may be further pressed and fixed toward the body.

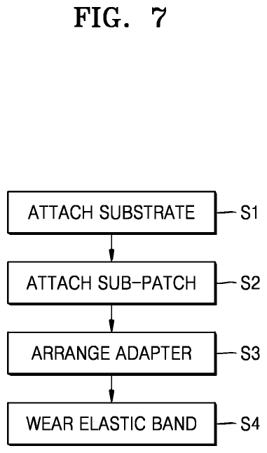

Hereinafter, an embodiment of a method of using the bio-signal measuring device 10 of the present disclosure will be described. The method of using the bio-signal measuring device 10 may be understood from the method described in FIG. 6 and the above, and summarized using FIG. 7 as follows. Hereinafter, terms defined in the method of using the bio-signal measuring device 10 have the same meaning as those used in the bio-signal measuring device 10 described above.

FIG. 7 is a flowchart of an embodiment of a method of using the bio-signal measuring device 10 of the present disclosure. Referring to FIG. 7, first, in the process of attaching the measuring device 100 in S1, the area to which the measuring device 100 is to be attached is checked, the release film 200 is removed, and the measuring device 100 is attached to the area to be attached. The attachment area of the measuring device 100 is below the left chest near the lung, and at this time, the housing 130 is oriented so as to come to the center of the chest. Before attaching the measuring device 100, it is useful to shave and disinfect with alcohol if necessary. After attaching the measuring device 100, it is useful to press the entire area evenly for at least 1 minute.

Then, in the process of attaching the sub-patch 300 in S2, after removing the protective film on the sub-patch 300, the sub-patch 300 is attached so that the sub-patch 300 covers the measuring device 100. The sub-patch 300 covers the measuring device 100 and the body.

Then, in the process of arranging the adapter 400 in S3, the tape of the adapter 400 is removed and a guide pad is attached on the sub-patch 300.

Then, in the process of wearing the elastic band 500 in S4, the elastic band 500 is worn at a position covering the measuring device 100. At this time, the elastic band 500 should be worn in an extended state so that the measuring device 100 may be further pressed and fixed toward the body. The elastic band 500 may be fixed by wearing it using buckles at both ends of the elastic band 500 or by attaching Velcro.

As described above, the present disclosure has been described with reference to the embodiment shown in the drawings, but this is only an example. Those of ordinary skill in the art may fully understand that various modifications and equivalent other embodiments are possible from the embodiments. Therefore, the true technical protection scope of the present disclosure should be determined based on the appended claims.

Certain technical content described in the embodiment is an embodiment and does not limit the technical scope of the embodiment. To concisely and clearly describe the description of the invention, descriptions of conventional general techniques and configurations may be omitted. In addition, the connection or connection members of lines between the components shown in the drawings are illustrative of functional connections and/or physical or circuit connections, and may be represented as a variety of functional connections, physical connections, or circuit connections that are replaceable or additional in an actual device. In addition, if there is no certain mention such as “essential” or “importantly”, it may not be an essential component for the application of the present disclosure.

In the description of the invention and in the claims, “above” or similar referents may refer to both the singular and the plural unless otherwise specified. In addition, when a range is described in the embodiment, it includes the invention to which individual values within the range are applied (unless there is a description to the contrary), and each individual value constituting the range is described in the description of the invention. In addition, the steps constituting the method according to the embodiment may be performed in an appropriate order unless the order is explicitly stated or there is no description to the contrary. The embodiments are not necessarily limited according to the order of description of the above steps. The use of all examples or illustrative terms (e.g., and the like) in the embodiment is merely for the purpose of describing the embodiment in detail, and unless limited by the claims, the scope of the embodiments is not limited by the above examples or exemplary terms. In addition, those skilled in the art will appreciate that various modifications, combinations, and changes may be made in accordance with design conditions and factors within the scope of the appended claims or their equivalents.

While the present disclosure has been particularly shown and described with reference to embodiments thereof, it will be understood that various changes in form and details may be made therein without departing from the spirit and scope of the following claims.

What is claimed is:

1. A bio-signal measuring device comprising:
a housing accommodating a battery;
a flexible substrate extending in a band shape, disposed on one side of the housing and electrically connected thereto, the flexible substrate including a plurality of sensor electrodes; and
at least one adapter formed to correspond to the housing and the flexible substrate, the adapter configured to press the flexible substrate,
wherein the housing and the plurality of sensor electrodes are disposed along a longitudinal direction of the flexible substrate.

2. The bio-signal measuring device of claim 1, the housing is disposed between the plurality of sensor electrodes at a position spaced apart from a center of the flexible substrate.

3. The bio-signal measuring device of claim 1, wherein the adapter further comprises a first pressing part disposed on one side of the housing, a second pressing part disposed on another side of the housing, and a connection part connecting the first pressing part to the second pressing part, wherein a housing groove into which the housing is inserted is formed between the first pressing part and the second pressing part.

4. The bio-signal measuring device of claim 3, wherein a thickness of the housing groove facing each inner end of the first pressing part and the second pressing part corresponds to a thickness of the housing.

5. The bio-signal measuring device of claim 3, wherein a thickness of each outer end of the first pressing part and the second pressing part gradually decrease relative to a thickness of each inner end of the first pressing part and the second pressing part.

6. The bio-signal measuring device of claim 3, wherein a shape of an upper end of the first pressing part and the second pressing part is in a straight line.

7. The bio-signal measuring device of claim 3, wherein the connection part is formed at a lower part of the housing groove.

8. The bio-signal measuring device of claim 2, further comprising an elastic band including an elastic material to press the flexible substrate, the adapter or both.

9. The bio-signal measuring device of claim 8, wherein the elastic band comprises a buckle device or Velcro fabric at both ends, so that the both ends of the elastic band are connected and detachable.

10. The bio-signal measuring device of claim 9, wherein the at least one adapter is embedded into the elastic band or integrated into the elastic band.

11. The bio-signal measuring device of claim 1, wherein a sensor electrode of the plurality of sensor electrodes comprises a capacitor sensor installed in the housing and configured to generate a respiration signal by sensing respiration and an ECG sensor installed in the flexible substrate and configured to generate an ECG signal by sensing a current or a voltage.

12. The bio-signal measuring device of claim 11, further comprising an alarm unit configured to generate an alarm through a lamp or speaker when a magnitude of the respiration signal or the ECG signal decreases.

13. The bio-signal measuring device of claim 1, further comprising at least one of an acoustic sensor configured to detect snoring or respiration during sleep and an acceleration sensor configured to detect a movement.

14. A bio-signal measuring device comprising:
a housing accommodating a battery;
a flexible substrate extending in a band shape, disposed on one side of the housing and electrically connected thereto, the flexible substrate including a plurality of electrodes; and
an adapter formed to correspond to the housing and the flexible substrate and disposed to press the flexible substrate.

15. A method of using a bio-signal measuring device, the method comprising:
attaching a substrate including a plurality of respiration sensors and a plurality of ECG measurement sensors to a level near a user's lung; and
placing a band including an elastic material to press an outer surface of the substrate toward the user's lung.

16. The method of claim 15, further comprising disposing an adapter formed in a shape corresponding to the substrate on an outer surface of the substrate before the placing the band.
